(19) United States
(12) Patent Application Publication
(10) Pub. No.: US 2007/0129444 A1
(43) Pub. Date: Jun. 7, 2007

(54) Novel weight reduction composition and uses thereof

(75) Inventor: Dennis J. Kalota, Fenton, MO (US)

Correspondence Address: Mallinckrodt Inc., 675 McDonnell Boulevard, HAZELWOOD, MO, 63042, US

(73) Assignee: Mallinckrodt Inc.

(21) Appl. No.: 11/633,698

(22) Filed: Dec. 4, 2006

Related U.S. Application Data
(60) Provisional application No. 60/742,598, filed on Dec. 6, 2005.

(51) Int. Cl. A61K 31/137; C07C 211/26

(52) U.S. Cl. 514/649; 564/373

(57) ABSTRACT

The present invention relates to a novel compound, N-benzylpropylhexedrine, and a process for preparing the compound. The invention also provides methods for reducing weight, decreasing appetite, inhibiting weight gain, and treating narcolepsy in patients by administering a pharmaceutical composition comprising N-benzylpropylhexedrine.

### FIELD OF THE INVENTION

The present invention relates to a novel compound N-benzylpropylhexedrine, and a weight reduction composition comprising the same. The present invention is also directed to a method for reducing weight, decreasing appetite, inhibiting weight gain or treating narcolepsy in a patient by administering a pharmaceutical composition comprising N-benzylpropylhexedrine.

### BACKGROUND OF THE INVENTION

In the last 20 years, there has been an increasing trend in obesity in the populations of the developed world. The increased incidence of obesity is due in part to the ready availability of food in numerous retail outlets and westernized diets that have high saturated fat and lower fiber contents such that the food is energy dense. The lifestyle of the populations of the developed world has also become more sedentary with the increased mechanization of society and the steady reduction of manual labor intensive industries. There now exists an energy imbalance between the energy intake from calorie dense foods and the reduced energy expenditure required for a sedentary lifestyle. Some of the excess energy intake is stored as fat in the adipose tissue, the accumulation of which over a period of time results in obesity and can be a significant contributory factor to other diseases and disorders.

Obesity is now recognized by the medical profession as a metabolic disease. In the USA, it is estimated that 25% of the adult population is considered clinically obese (Body Mass Index>30). Such obesity has caused or contributed to marked increases in the occurrence of heart disease, hypertension, diabetes, osteoarthritis of the knees and hips, and increased morbidity resulting from related medical conditions. It has been estimated that $45 billion of US healthcare costs, or 8% per annum of total healthcare expenditures, is as a direct result of obesity. The traditional approaches to long term weight management such as diet and exercise have proved ineffective alone to control the spread of obesity. Today, more than ever, there is considerable interest in developing safe, effective drugs for the treatment of obesity.

Pharmacological approaches to the treatment of obesity have focused on either developing drugs that increase energy expenditure or drugs that reduce energy intake.

During the past five decades several medications have been tried with patients to reduce weight. Sibutramine (Meridia®) also acts on the central nervous system but has as side effects headache, insomnia, chest palpitations, hypertension, and dry mouth. Another type of weight loss medication is orilistat (Xenecal®), which acts by inhibiting the absorption of fat in the small intestine. This medication has as side effects oily stool, increased flatus, and occasional stool incontinence. Supplemental fat soluble vitamins (A, D, and E) must be given to avoid a deficiency of these vitamins caused by the medication.

Other exemplary treatments are disclosed in U.S. Pat. No. 3,867,539 (administration of histidine); U.S. Pat. No. 4,446,138 (administration of L-Dopa); U.S Pat No. 4,588,724 (administration of beta adrenergic stimulant or alpha-2 adrenergic inhibitor); U.S. Pat. No. 4,745,122 (administration of paroxetine); U.S. Pat. No. 5,019,594 (sympathomimetic drug and tyrosine); U.S. Pat. No. 5,300,298 (administration of 8-phenylxanthines); U.S. Pat. No. 5,403,851 (tryptamine); U.S. Pat. No. 5,567,714 (administration of neuropeptide Y); U.S. Pat. No. 5,573,774 (nicotinic metabolites); and U.S. Pat. No. 5,578,613 (administration of 2-phenyl-3-aroylbenzothiophenes).

Amphetamines were also used in the past to treat obesity. The amphetamine family of central nervous system (CNS) stimulants includes amphetamine, methamphetamine, and dextroamphetamine, among others. Amphetamines act on the brain by stimulating the release of norepinephrine and dopamine at the nerve synapses. The amphetamines are part of a broader class of compounds known as the phenethylamines, a class which includes a broad array of pharmacologically active compounds, such as ephedrine, mescaline, phentermine, and fenfluramine, among others. However, due to the serious side effects that can accompany the use of amphetamines, they have been discontinued in the treatment of obesity.

Sympathomimetic amines, such as benzphetamine hydrochloride, are similar in effects to amphetamines, but differ from amphetamines in that they have little or no effect on dopamine release at the synapse. Benzphetamine hydrochloride, which is a potent anorectic (also known as “anorexigenic” or appetite suppressant), has found use in a variety of applications. It is commercially sold as DIDREX® tablets, which are indicated for the management of exogenous obesity as a short term adjunct (a few weeks) in a regimen of weight reduction based on caloric restriction. The suggested dosage ranges from 25 to 50 mg one to three times daily. However, the administration of benzphetamine hydrocholoride, similarly to other drugs in the symphatomimetic amine class, results in many cases in tachyphylaxis and tolerance. Considering the efficacy of benzphetamine hydrochloride in achieving weight loss, and the increasing incidence of obesity throughout the world, it is important to develop new compositions and methods for the treatment of obesity.

### SUMMARY OF THE INVENTION

The present invention provides a novel compound, N-benzylpropylhexedrine. This compound can be used to reduce weight, suppress appetite, inhibit weight gain or treat narcolepsy in patients. While not being bound to a particular theory, it is believed that N-benzylpropylhexedrine may provide increased and/or longer-lasting activity compared to benzphetamine, which is structurally related thereto.

Therefore, it is one aspect of the present invention to provide a weight reduction composition comprising N-benzylpropylhexedrine. In one aspect, N-benzylpropylhexedrine is S-N-benzylpropylhexedrine. In another aspect, the N-benzylpropylhexedrine is the racemic form (R,S) of N-benzylpropylhexedrine. Such composition can be formulated with different pharmaceutically acceptable carriers or excipients. In a preferred embodiment, a pharmaceutical composition comprising N-benzylpropylhexedrine is administered orally. In addition, N-benzylpropylhexedrine can be administered in combination with other obesity treatments, such as behavior therapy, calorie-restricted diet, exercise regimen, and/or other pharmaceutical agents.

With respect to the obesity treatments, the present invention provides methods of decreasing appetite, reducing weight, and/or inhibiting weight gain in a patient by administering a pharmaceutical composition comprising N-benzylpropylhexedrine.

It is another aspect of the invention to provide a method of treating narcolepsy in a patient by administering a pharmaceutical composition comprising N-benzylpropylhexedrine.

Other objects and features will be in part apparent and in part pointed out hereinafter.

### DETAILED DESCRIPTION OF THE INVENTION

Among the aspects of the present invention is the provision of a novel compound, namely N-benzylpropylhexedrine.

N-benzylpropylhexedrine is structurally related to benzphetamine, as indicated in their structures shown below:

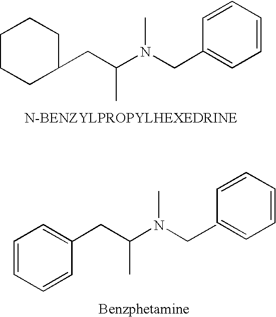

Benzphetamine was also indicated for the treatment of narcolepsy; however, due to its high abuse potential, many patients were prescribed a weaker stimulant, propylhexedrine.

Propylhexedrine is an adrenergic compound, used as a vasoconstrictor to decongest nasal mucosa due to colds, allergies and allergic rhinitis. It is generally administered by inhalation. Propylhexedrine is also produced in a significant amount in a conversion of methamphetamine to benzphetamine, which is indicated for the management of obesity for a short term period, e.g., a few weeks.

Therefore, it is believed that N-benzylpropylhexedrine is useful for treatment of obesity and narcolepsy. In one aspect, it is the S isomer of the N-benzylpropylhexedrine that is used. In another embodiment, it is the racemic (R,S) form of N-benzylpropylhexedrine that is used.

N-benzylpropylhexedrine utilized in the present invention may be in the form of free bases or pharmaceutically acceptable acid addition salts thereof. The term “pharmaceutically-acceptable salts” are salts commonly used to form alkali metal salts and to form addition salts of free acids or free bases. The nature of the salt can vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methylglucamine) and procaine. All of these salts may be prepared by conventional means from the corresponding compound by reacting, for example, the appropriate acid or base with the compound of any Formula set forth herein.

N-benzylpropylhexedrine can be formulated into pharmaceutical compositions utilizing a number of different pharmaceutically acceptable carriers or excipients, and administered by a number of different means that will deliver a therapeutically effective dose. Such compositions can be administered orally, parenterally, by inhalation spray, rectally, intradermally, transdermally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures but liquid at the rectal temperature, and which will therefore melt in the rectum and release the drug.

In a preferred embodiment, a pharmaceutical composition comprising N-benzylpropylhexedrine is administered orally. Solid dosage forms for oral administration may include capsules, tablets, pills, powders, and granules. In such solid dosage forms, the compounds are ordinarily combined with one or more adjuvants appropriate to the indicated route of administration. If administered orally, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of active ingredient that can be combined with the carrier materials to produce a single dosage of the N-benzylpropylhexedrine will vary depending upon the patient and the particular mode of administration. In general, the pharmaceutical compositions may contain N-benzylpropylhexedrine in the range of about 10 mg to about 150 mg, more typically, in the range of about 20 mg to about 100 mg and still more typically, between about 25 mg and about 50 mg. A daily dose of about 10 mg to about 150 mg, more typically of about 20 mg to about 100 mg may be appropriate. The daily dose is generally administered in one to about four doses per day.

Generally, N-benzylpropylhexedrine is administered to a patient for a period of about 1 week to about 50 weeks. More typically, N-benzylpropyihexedrine is administered for about 10 weeks to about 30 weeks.

As a weight reduction composition, N-benzylpropylhexedrine may be used in several different therapeutic methods. It may be used to decrease appetite, reduce weight or inhibit weight gain in a patient. All of these methods are useful as part of obesity treatment. N-benzylpropylhexedrine may be indicated as the only therapy for obesity, or it may be given in conjunction with additional therapies. By way of example, the additional obesity therapies may include behavior therapy, calorie-restricted diets, exercise regimens, weight loss surgery or additional drug therapies. For example, N-benzylpropylhexedrine may be administered in combination with one or more of the following drugs: sibutramine, orlistat, mazindol, diethylpropion, phentermine, and phendimetrazine. Such combination therapy may be administered simultaneously or the drugs may be given separately. For example, if the additional drug is orlistat, the daily dosage of N-benzylpropylhexedrine may be between about 10 mg and 150 mg, and the daily dosage of orlistat may be between 100 mg and 400 mg. One skilled in the art can readily determine the daily dosages and frequency of administration for such combinations. As another example, N-benzylpropylhexedrine may be given to patients who are undergoing behavior therapy or have started an exercise regimen.

In addition, a pharmaceutical composition comprising N-benzylpropylhexedrine may be used to treat narcolepsy. Narcolepsy is a sleep disorder characterized by excessive daytime sleepiness. Other symptoms include uncontrollable episodes of sleep, loss of muscle control (cataplexy), sleep paralysis, hypnagogic hallucinations, and automatic behavior. There are two classes of medications generally used to treat narcolepsy. The first class includes stimulants, which are used to promote daytime alertness, whereas the other class includes anti-depressants, which are used to treat cataplexy, hypnagogic hallucinations and sleep paralysis. Stimulants such as methylphenidate (Ritalin®), dextroamphetamine (Dexedrine®) and pemoline (Cylert®) are used to combat hypersomnia and nap attacks. Modafinil (Provigil®) is a wake-promoting drug that helps prevent hypersomnia without stimulating the rest of the body. It has done well in studies in improving daytime alertness without the same side effects and addictive qualities found in other stimulants. Among anti-depressants, the multicyclics, such as imipramine, desipramine, clomipramine, and protriptyline and selective serotonin reuptake inhibitors, such as fluoxetine and sertraline are the most commonly prescribed. Xyrem is a hypnotic and it can improve the quality of nighttime sleep by reducing the possibility of nighttime awakenings. During the day, it also reduces the likelihood of cataplexy.

Stimulants can be accompanied by serious side effects, such as addiction and abuse. Furthermore, tolerance to these drugs can develop, which is why it is advised for patients taking stimulants to undergo drug holidays (such as to withdraw gradually from the drug and resume treatment at a lower dose). N-benzylpropylhexadrine, which exhibits weaker potency than the traditional stimulants is, therefore, believed to be effective in treating narcolepsy while avoiding some of the side effects. It can be administered as a single therapy, or it can be administered in combination with anti-depressants. One skilled in the art can determine for each patient the right dosage and administration regimen, based on the factors such as the patient's medical history, seriousness of disorder, and age.

In addition, N-benzylpropylhexedrine may be administered as part of a combination treatment for narcolepsy. By way of example, N-benzylpropylhexedrine can be administered in conjunction with an anti-depressant, such as imipramine, desipramine, clomipramine, or protriptyline, wherein the combination can be administered either simultaneously or at different times. For example, a patient suffering from narcolepsy may be administered daily dosages of between about 10 mg and 150 mg of N-benzylpropylhexedrine and between about 50 mg and 300 mg of imipramine. One skilled in the art can readily determine the appropriate combinations of drugs and dosages based on a particular patient.

In one embodiment, this compound is derived from the reaction of propylhexedrine and a benzyl halide. In another embodiment, the benzyl halide is benzyl chloride. A detailed method of N-benzylpropylhexedrine synthesis is described in Examples 1-4. Examples 1 and 2 describe the preparation of the S isomer of N-benzylpropylehexedrine, Example 3 describes the preparation of the racemic form, and Example 4 describes the synthesis of the R isomer. Depending on whether the synthesis is started with S, R, or the racemic (R,S) form of propylhexedrine, the reaction will yield the S, R, or (R,S) form of the N-benzylpropylhexedrine. Examples of methods for making S-propylhexedrine (d-propylhexedrine), R-propylhexedrine (l-propylhexedrine) and racemic propylhexedrine can be found in, e.g., U.S. Pat. No. 3,014,966. Specifically, see examples I through V. S-propylhexedrine (d-propylhexedrine) is described in example III. Example IV describes making the R isomer (l-form) of propylhexedrine, whereas examples I and II describe making the racemic form.

Generally, N-benzylpropylhexedrine can be prepared by mixing propylhexedrine with toluene, sodium carbonate and benzyl halide in the presence of heat. The mixture is next extracted with an organic solvent such as toluene to separate the N-benzylpropylhexdrine from the inorganic salts, neutralized with an acid such as hydrochloric acid to convert the base into the salt form, dried, and concentrated by distillation, following which the distillation residue is cooled to afford a crystallized mass. The crystals of N-benzylpropylhexedrine are next isolated using vacuum filtration or centrifugation.

Abbreviations and Definitions

To facilitate understanding of the invention, a number of terms are delined below.

The term “pharmaceutically acceptable” is used adjectivally herein to mean that the modified noun is appropriate for use in a pharmaceutical product; that is the pharmaceutically acceptable material is relatively safe and/or non-toxic, though not necessarily providing a separable therapeutic benefit by itself.

The term “patient” includes any human or animal. The animal can be a domestic livestock species, a laboratory animal species, or a zoo animal. The terms “patient” and “subject” are used interchangeably herein.

The term “therapeutically-effective” is intended to qualify the amount of an agent or combination of two or more agents, which will achieve the goal of improvement in disorder severity and/or the frequency of incidence over no treatment.

The term “treatment” includes alleviation, elimination of causation of or prevention of undesirable symptoms associated with a disease or disorder. Treatment as used herein includes prophylactic treatment.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

The following examples illustrate the invention, but are not to be taken as limiting the various aspects of the invention so illustrated.

### EXAMPLES

### Example 1

### Synthesis of S-N-benzylpropylhexedrine

To a 100 ml 3-neck flask equipped with a mechanical stirrer and a thermometer was added 4.14 g (0.0272 mole) of propylhexedrine, 7.83 g of toluene, 6.23 g (0.0383 mole) of sodium carbonate, and 3.56 g (0.0278 mole) of 99% benzyl chloride. The stirred reaction mass was heated to 116° C. for five hours, following which 12.5 ml of toluene and 40 ml of water was added to the reaction mass. The mixture was vigorously stirred and then transferred to a separatory funnel. The toluene and water layers were separated affording 43.09 g of water layer. The toluene layer was washed with 10 ml of water. The toluene and water layers were separated affording 26.69 g of toluene layer that contained the N-benzylpropylhexedrine. A 3.25 g sample of the toluene layer was taken for analysis. To the remaining toluene layer was added 2.58 g (0.0265 mole) of 37.5% hydrochloric acid. After agitating the mixture was transferred to a flask equipped with a Dean-Stark trap and condenser, followed by the addition of 100 ml of toluene. The azeotropic distillation was continued until the distillate was clear which indicated that the water was removed. Next, most of the toluene was distilled forward. The distillation residue was cooled overnight affording a crystallized mass. These crystals were isolated by vacuum filtration and washed with toluene affording 4.98 g (0.0176 mole, 69% yield) of N-benzylpropylhexedrine having a melting point of 145.0-147.7° C.

### Example 2

### Synthesis of S-N-benzylpropylhexedrine

To a 100 ml 3-neck flask was added 1.95 g (0.0125 mole) of propylhexedrine and 2.45 g of toluene. To this mixture was added 0.79 g (0.00624 mole) of benzyl chloride. The one equivalent excess of propylhexedrine was used to scavenge the hydrogen chloride that was generated during the benzylation. The mixture was stirred and the flask was purged with nitrogen, and then heated to 124° C. for 3.25 hours. A mixture of a solid (propylhexedrine HCl) and an oil formed. To the mixture was added 4.60 g of toluene and 7.32 g of water. The mixture was aggressively stirred and heated to 50° C. for 10 minutes. The two liquid layers were transferred to a separatory funnel. The 3-neck flask was rinsed with 0.85 g of water and 1.62 g of toluene. The toluene and water layers were separated into a 9.10 g water layer that contained the propylhexedrine hydrochloride and an 8.19 g toluene layer that contained the N-benzylpropylhexedrine. Analysis of the toluene layer by HPLC found that it contained 1.70 g (0.00692 mole, 110% yield) of N-benzylpropylhexedrine. To the water layer was added 5.68 g of toluene and 1.18 g of 25% sodium hydroxide solution. The mixture was aggressively agitated and the layers were separated into a 9.05 g water layer having a pH of 13 and a 6.79 g toluene layer. Analysis of the toluene layer found 0.8895 g of propylhexedrine.

### Example 3

### Synthesis of (RS)-N-benzylpropylhexedrine

The (RS)-N-benzylpropylhexedrine is made from (RS)-propylhexedrine by the same route as the (S)-N-benzylpropylhexedrine was made in example 1. For example, to a 100 ml 3-neck flask equipped with a mechanical stirrer, thermometer, and a connection for applying a continuous nitrogen purge is added 4.14 g (0.0272 mole) of (RS)-propyhexedrine, 7.83 g of toluene 6.23 g (0.0383 mole) of sodium carbonate, and 3.56 g (0.0278 mole) of benzyl chloride. A continuous nitrogen purge is applied. The stirred reaction mass is heated to 116° C. for five hours, following which 12.5 ml of toluene and 40 ml of water are added to the reaction mass. The mixture is vigorously stirred and then transferred to a separatory funnel. The toluene and water layers are separated affording 43.09 g of water layer. To the toluene layer in the separatory funnel is added 10 ml of water. The mixture is vigorously agitated and then the water and toluene layers are separated affording 26.69 g of toluene layer containing the (RS)-N-benzylpropylhexedrine. To the toluene layer is added 2.94 g of 37% hydrochloric acid and the mixture is vigorously agitated. The mixture is transferred to a flask equipped with a Dean-Stark trap and condenser, followed by the addition of 100 ml of toluene. The azeotropic distillation is continued until the distillate is clear which indicates that the water was removed. Next, most of the toluene is distilled forward. The distillation residue is cooled affording a crystallized mass. The crystals are isolated by vacuum filtration and washed with toluene affording 5.6 g (0.0199 mole) of d,1-N-benzyipropylhexedrine.

### Example 4

### Synthesis of (R)-N-benzylpropylhexedrine

The R-N-benzylpropylhexedrine is made from (R)-propylhexedrine (1-N-methyl-1-cyclohexylisopropylamine) by the same route as the d-N-benzylpropylhexedrine was made in example 1. For example, to a 100 ml 3-neck flask equipped with a mechanical stirrer, thermometer, and a connection for applying a continuous nitrogen purge is added 4.14 g (0.0272 mole) of (R)-propyhexedrine, 7.83 g of toluene 6.23 g (0.0383 mole) of sodium carbonate, and 3.56 g (0.0278 mole) of benzyl chloride. A continuous nitrogen purge is applied. The stirred reaction mass is heated to 116° C. for five hours, following which 12.5 ml of toluene and 40 ml of water are added to the reaction mass. The mixture is vigorously stirred and then transferred to a separatory funnel. The toluene and water layers are separated affording 43.09 g of water layer. To the toluene layer in the separatory funnel is added 10 ml of water. The mixture is vigorously agitated and then the water and toluene layers are separated affording 26.69 g of toluene layer containing the 1-N-benzylpropylhexedrine. To the toluene layer is added 2.94 g of 37% hydrochloric acid and the mixture is vigorously agitated. The mixture is transferred to a flask equipped with a Dean-Stark trap and condenser, followed by the addition of 100 ml of toluene. The azeotropic distillation is continued until the distillate is clear which indicates that the water was removed. Next, most of the toluene is distilled forward. The distillation residue is cooled affording a crystallized mass. The crystals are isolated by vacuum filtration and washed with toluene affording 5.6 g (0.0199 mole) of 1-N-benzylpropylhexedrine.

What is claimed is:

1. N-benzylpropylhexedrine, having the formula:

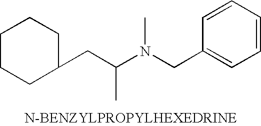

2. A weight reduction composition comprising N-benzylpropylhexedrine and a pharmaceutically acceptable carrier or excipient.

3. The weight reduction composition of claim 2, wherein the N-benzylpropylhexedrine is (S)-N-benzylpropylhexedrine.

4. The weight reduction composition of claim 2, wherein the N-benzylpropylhexedrine is (R,S)-N-benzylpropylhexedrine.

5. The weight reduction composition of claim 2, wherein the composition comprises from about 10 mg to about 150 mg of N-benzylpropylhexedrine.

6. The weight reduction composition of claim 5, wherein the composition comprises from about 20 mg to about 100 mg of N-benzylpropylhexedrine.

7. The weight reduction composition of claim 2, wherein the composition is an oral formulation.

8. The weight reduction composition of claim 7, wherein the composition is in a form of a tablet, capsule, pill or suspension.

9. The weight reduction composition of claim 2, wherein the composition is administered to a patient for a period of about 1 week to about 50 weeks.

10. The weight reduction composition of claim 2, wherein the composition is administered to a patient from once a day to four times a day.

11. The weight reduction composition of claim 2, wherein the composition further comprises a therapeutic agent selected from the group consisting of sibutramine, orlistat, mazindol, diethylpropion, phentermine, and phendimetrazine.

12. A method of reducing weight in a patient, wherein the method comprises administering to a patient a pharmaceutical composition comprising N-benzylpropylhexedrine and a pharmaceutically acceptable carrier or excipient.

13. The method of claim 12, wherein the N-benzylpropylhexedrine is (S)-N-benzylpropylhexedrine or (R,S)-N-benzylpropylhexedrine.

14. The method of claim 12, wherein the pharmaceutical composition comprises from about 10 mg to about 150 mg of N-benzylpropylhexedrine.

15. The method of claim 12, wherein the pharmaceutical composition is an oral formulation.

16. The method of claim 12, wherein the pharmaceutical composition further comprises a therapeutic agent selected from the group consisting of sibutramine, orlistat, mazindol, diethylpropion, phentermine, and phendimetrazine.

17. A method of treating narcolepsy in a patient, wherein the method comprises administering to a patient a pharmaceutical composition comprising N-benzylpropylhexedrine and a pharmaceutically acceptable carrier or excipient.

18. The method of claim 17, wherein the N-benzylpropylhexedrine is (S)-N-benzylpropyihexedrine or (R,S)-N-benzylpropylhexedrine.

19. The method of claim 17, wherein the pharmaceutical composition comprises from about 10 mg to about 150 mg of N-benzylpropylhexedrine.

20. The method of claim 17, wherein the pharmaceutical composition is an oral formulation.

21. The method of claim 17, wherein the pharmaceutical composition further comprises a therapeutic agent selected from the group consisting of imipramine, desipramine, clomipramine, and protriptyline.

22. A method of making N-benzylpropylhexedrine comprising reacting propylhexedrine with a benzyl halide.

23. The method of claim 22, wherein the benzyl halide is benzyl chloride.

24. The method of claim 22, wherein the N-benzylpropylhexedrine is (S)-N-benzylpropylhexedrine or (R,S)-N-benzylpropylhexedrine.
